(19) United States
(12) Patent Application Publication
(10) Pub. No.: US 2006/0085028 A1
(43) Pub. Date: Apr. 20, 2006

(54) Vessel occlusion system

(76) Inventor: Robert Boock, San Diego, CA (US)

Correspondence Address: SCHWEGMAN, LUNDBERG, WOESSNER & KLUTH, 1600 TCF TOWER, 121 SOUTH EIGHT STREET, MINNEAPOLIS, MN, 55402, US

(21) Appl. No.: 10/967,661

(22) Filed: Oct. 18, 2004

(51) Int. Cl. A61M 29/00

(52) U.S. Cl. 606/200

(57) ABSTRACT

The present invention relates to a device for occluding vessels or ducts in a living being. The device includes a frame for anchoring the device in a vessel and a expandable core connected to the frame for occluding the vessel.

### BACKGROUND OF INVENTION

The inventive subject matter described herein relates to a vessel occlusion device and to a method for occluding a vessel or other duct in a living being.

Endovascular therapy has been used to treat conditions such as internal bleeding, tumor growth, and vessel wall pressure in a region of aneurysm. Endovascular therapy has included a step of occluding blood supply to tumors and relieving vessel wall pressure in a region of vessel aneurysm. Endovascular therapy has included mechanically based therapy as well as chemically based therapy. Catheters have played a significant role in performing endovascular therapy.

One challenge in successful endovascular therapy is that a target site which requires treatment may be in a region of an organism, such as the brain, which requires catheter placement along a tortuous path that includes small vessels or ducts, such as arterial vessels.

One method for performing therapeutic embolization procedures, such as is described in the Gianturco patent, U.S. Pat. No. 5,334,210, issuing Aug. 2, 1994, employs a detachable, inflatable balloon formed of a material such as latex or silicone. During an embolization procedure, the detachable balloon is attached to a distal end of a delivery catheter and positioned at a treatment site using a visualization aide such as fluoroscopy. Once positioned, the balloon is filled with a solidifying gelatinous fluid or contrast media. Secure anchoring is then tested. If necessary, the balloon is then additionally filled and mechanically detached from the delivery catheter. The gelatinous fluid solidifies and the balloon occludes the blood vessel at the treatment site.

The second type of mechanical vaso-occlusive device is a wire coil which can be introduced through a catheter in a stretched linear form and which assumes a helical wire shape when released into a vessel. The wire itself tends to be relatively stiff and shape retaining and typically made of platinum or stainless steel in a coil shape. The wire is sometimes coated with filaments such as Dacron® or cotton fibers which provide a substrate for clot formation in an interior region of a vessel while the coil serves to anchor the device on the vessel wall at a site of release.

U.S. Pat. No. 5,639,277 issuing Jun. 17, 1997, describes a surgical device for forming a vessel occlusion or embolism. The device is a helically wound coil in which a helix is wound in such a way as to have multiple axially offset longitudinal or focal axes. The device also includes small diameter secondary coil windings that are adjacent large diameter coil windings. The device is sufficiently flexible and small that it may be delivered to a site within vessels or ducts of the human body using a pusher and a catheter.

U.S. Pat. No. 5,536,274, issuing Jul. 16, 1996, describes a spiral implant for blood vessels. The implant is advanced to a desired site in a living being by advancement of a catheter in which the implant is positioned. The implant is displaced from the catheter by displacement of an insertion wire upon which the implant is positioned. The implant is displaced in an extended shape to the intended site for location. The implant is formed into a secondary shape by withdrawing the guidewire or by pushing forward a stripping element. Once the implant is in a desired position, the implant is stripped off the guidewire using the stripping element.

U.S. Pat. No. 4,994,069, issuing Feb. 19, 1991, describes a vessel-occlusion coil wire device. The device has a relaxed condition in which the wire assumes a folded, convoluted conformation, a stretched condition. The wire also has a stretched condition so that the wire can be pushed through a catheter. The wire additionally has a memory which returns the wire from its stretched condition to its relaxed condition. When the wire is released from the catheter, the wire forms a vaso-occlusive body.

### SUMMARY OF THE INVENTION

The invention described herein includes a system for occluding vessels or ducts in a living being. The system includes a frame and an expandable core. The frame secures the system to a vessel in a living being. The expandable core expands to occlude the vessel.

In an embodiment, the frame is a stent, which has a collapsed position for insertion through a vessel to an occlusion site and an expanded position for securing the stent to the vessel wall. The frame anchors and stabilizes the occlusion device once it is positioned in a vessel or duct.

In an embodiment, the expandable core expands at the occlusion site to occlude the vessel or duct. The expandable core may include a hydrogel, polyurethane foam, or a combination of these materials. In an embodiment, the expandable core is coated to control the rate of expansion of the core. In an embodiment, the core is a generally solid body that blocks fluid flow in a vessel. In an embodiment, the expandable core is biodegradable such that after a period of time the core degrades and fluid flow in the vessel resumes.

The present invention also includes a method for occluding vessels or ducts in a living being. The method includes providing a device comprising a frame and an expandable core. The frame is positioned at an occlusion site and secured to a vessel wall. The expandable core expands to occlude the vessel. The method may also include transporting the device to the occlusion site. The device is transported in a compressed or collapsed state relative to its occlusion state. Once at the site of occlusion, the device expands and secures itself in the vessel or duct. In an embodiment, the device is retrieved from the vessel, for example by a transporting unit, thereby returning the vessel to its original state.

An embodiment of the present invention includes a method of treating a living being which provides a therapeutic delivery component through the occlusion device.

The therapeutic delivery component provides medicines, chemotherapeutics, or other drugs to a treatment site downstream of the occlusion site.

### DESCRIPTION OF THE DRAWINGS

FIG. 1 illustrates an elevational view of one embodiment of the vessel occlusion system of the present invention.

FIG. 2 illustrates another elevational view of the vessel occlusion system of the present invention.

FIG. 3 illustrates another elevational view of the vessel occlusion system of the present invention.

FIG. 4 illustrates a cross sectional view taken generally along line 4-4 of FIG. 2.

FIG. 5 illustrates the vessel occlusion device in its use position in a vessel.

FIG. 6 illustrates an embodiment of the vessel occlusion system of the present invention.

FIG. 7 illustrates an embodiment of the vessel occlusion system of the present invention.

FIG. 8 illustrates a method according to the present invention.

### DETAILED DESCRIPTION OF PREFERRED EMBODIMENTS

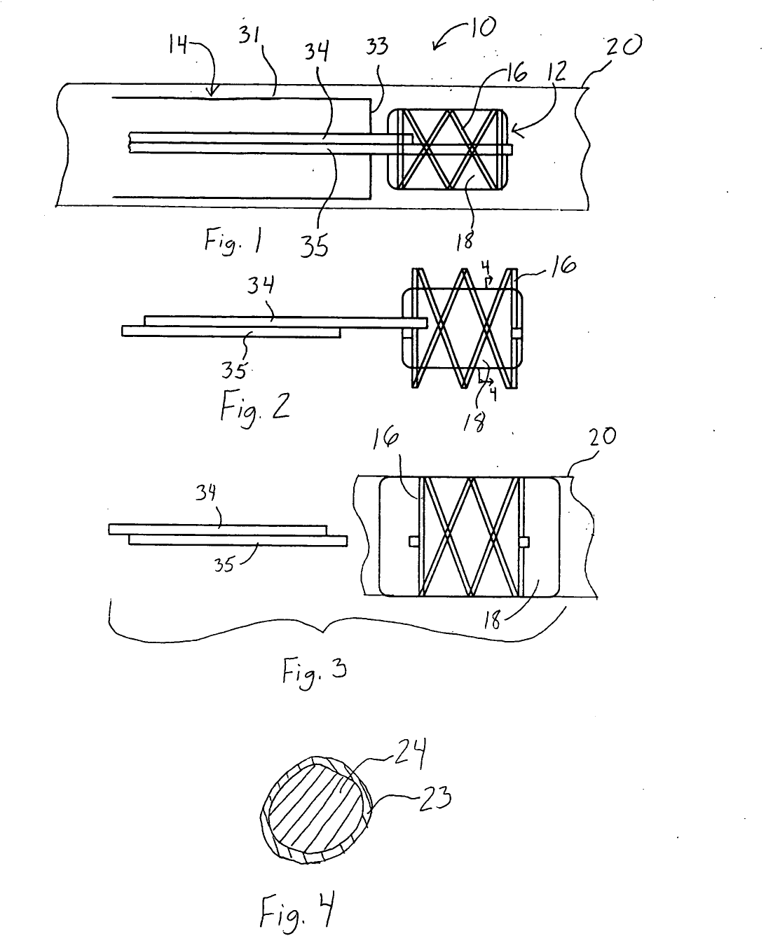

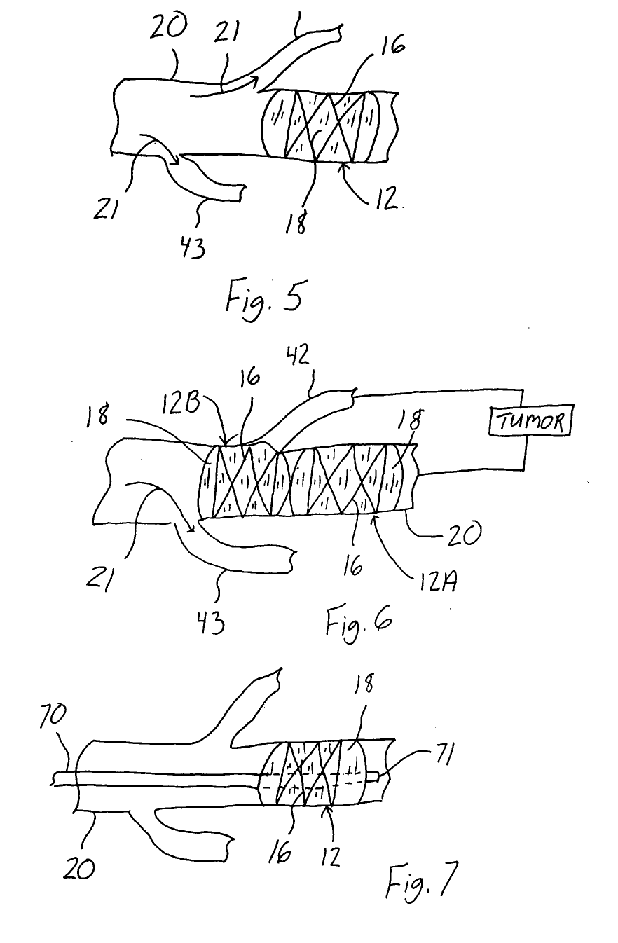

The occlusion system of the invention described herein, illustrated in one embodiment generally at 10 in FIG. 1, includes an occlusion device 12 and a delivery or transport unit 14 removably connected to the occlusion device 12. Delivery unit 14 includes a catheter 31 and two control wires 34 and 35. Delivery unit 14 is adapted to transport the occlusion device 12 to an occlusion site in a vessel and to release the occlusion device at the occlusion site. The occlusion device 12 has a frame 16 and an expandable core 18 secured to the frame 16. For some embodiments, core 18 is secured to frame 16 by an adhesive or glue. In one embodiment, core 18 is secured to frame 16 by being formed, e.g., molded, around at least part of the frame.

The present description uses the term “vessel” to describe a tube, duct or other pathway. One example of a vessel is a blood vessel in a living being such as a person or an animal. Other examples include any tubular channel, sometimes in an enclosed system, that conducts a bodily fluid such as blood, glandular secretions, or other bodily fluids.

Frame 16 has a collapsed, transport state as shown in FIG. 1. The core 18 has a nonexpanded, non-occlusion state as shown in FIG. 1. With both the frame 16 in the collapsed state and the core 18 in its nonexpanded state, the occlusion device 12 is positioned at an occlusion site in a vessel or duct 20 of a living being using the delivery unit 14. Fluid flow in the vessel 20 is generally in the direction of arrow 21.

Frame 16 is a wire construction that has a memory of a non-collapsed, non-transport state (FIGS. 2 and 3). Thus, when the frame is released from the delivery unit 14 at an occlusion site, frame 16 increases in diametrical size such that the frame 16 is secured to the vessel wall, for example by radial pressure exerted by the frame attempting to return to its full memory size and being restricted by the vessel wall. The frame 16 exerts sufficient radially outward pressure to anchor the frame in place but not damage the vessel 20. In one embodiment, the frame 16 has a compressed, collapsed cross-section diameter of about 0.020 inches and an expanded diameter of about 4.0 millimeters. The frame 16 has a length of about three-quarters of a centimeter in the collapsed state and a length of about five millimeters in the non-collapsed state. In an embodiment, the expanded state length is less than five millimeters.

While in the collapsed position, the frame 16 is transported to the occlusion site through a catheter 31 of the delivery unit 14. In an embodiment, the frame is self-expanding so that the device naturally expands from a collapsed state to a non-collapsed state at the occlusion site in order to release a natural tension from compression or in response to a memory imparted to the wire frame. In another embodiment, the frame 16 is held in the collapsed state until a positive force is applied to the frame 16 to cause it to expand to the non-collapsed state and engage the vessel wall.

In one embodiment, the frame 16 is a medical stent formed of interconnected wires. The term “wire” used herein may refer to a filament, unitary wire or a braid used in frame 16. The wire may have a filament diameter of about 0.005 inches or less. In one embodiment the wire diameter is about 0.003 inches. The wire may be generally flat, square, round, half-round, or triangular in cross-section. It will be understood that other embodiments of the wire may have other polygonal cross sections. The filament or wire may be made from biocompatible materials. Examples of biocompatible materials include metals, alloys, polymers, and the like. The wire may include one of platinum, palladium, rhodium, gold, silver, tungsten, iridium, nickel-titanium alloys, Elgiloy, and various stainless steels as well as materials coated with a biocompatible coating. Alloys of the listed metals are also suitable. Suitable biocompatible polymers for use as wire or filament include polyethylene, polyurethane, polyester, and polypropylene. It is also believed that polymers such as nylon, Teflon®, and inorganic materials such as fibrous carbon are also suitable for use in the present invention.

The wire or braid is woven to make a cross-hatch or mesh pattern in an embodiment. It is important that wires or fibers in the pattern have a capacity to slidably move over each other in order to render the frame 16 having the first collapsed, transport position and the second non-collapsed, anchor position. In one embodiment, wire or filament is woven at about 40 to 120 pics per inch. Other types of wire or fibers that have sufficient rigidity and strength to resist deformation when and while inserted in a vessel, such as a blood vessel, are also with the scope of the present invention.

Core 18 includes an interior portion 21, which is a expandable material that occludes a vessel in its expanded, occlusion state (FIG. 3). In an embodiment, the expandable interior portion of the core includes hydrophillic materials. In an embodiment, the expandable interior portion of the core includes gels. In an embodiment, the expandable interior portion of the core includes hydrophillic macromolecules. In an embodiment, the expandable interior portion of the core includes hydrogels. In an embodiment, the expandable interior portion of the core includes polyurethane. It is also within the scope of the present invention to provide a core with a combination of at least two of hydrophillic materials, hydrophillic macromolecules, gels, hydrogels, and polyurethane. The core interior portion material that travels into the vessel and expands to occlude a vessel are within the scope of the present invention.

In general, materials for the interior portion 21 include a material that has a small transport size and that grows into a enlarged occlusion size. In an embodiment, the interior portion 21 self expands by absorbing moisture present in the vessel into its structure. One example of such a material is a solid of a colloidal system in which a liquid material, such as water, blood, or other bodily fluid is dispersed. In an embodiment, the core interior portion 21 is biodegradable such that after it expands to occlude a vessel the core interior portion will degrade and stop occluding the vessel.

Accordingly, in this embodiment, the core 18 will occlude the vessel for a time period and after expiration it will degrade to allow fluid flow through the vessel without the need for surgical intervention to remove the occlusion device 12. Thus, the core 18 provides a temporary occlusion with resorption of the core into the living being.

If desired, the frame 16 may be subsequently removed by a surgical procedure. However, the frame 16 may be left in the vessel to support it. Consequently, the occlusion device 12 temporarily occludes the vessel to enhance drug retention downstream of the occlusion site as well as ischemically treating areas of the living being downstream of the occlusion site. In an embodiment, the core 18 occludes the vessel for at least about an hour. In an embodiment, the core 18 occludes the vessel for less than several hours.

In an embodiment, the core occludes the vessel for less than about six hours. In an embodiment, the core occludes the vessel for less than about twelve hours. In an embodiment, the core occludes the vessel for less than about eighteen hours. In an embodiment, the core 18 occludes the vessel for at least about a day. In an embodiment, the core 18 occludes the vessel for less than several days. In an embodiment, the core 18 occludes the vessel for at least about two days. In an embodiment, the core occludes the vessel for less than a week. The time period for occluding the vessel is selected based on the therapy required to treat the living being.

The core 18, as shown in the figures has a generally cyclindrical shape. It is within the scope of the present invention to form the core in any shape that will assist in it completely occluding a vessel. For example, the core 18 may be shaped as a helical, coil or spiral. Other embodiments of the core 18 include frustum, cone or pyramid shapes. Core 18 may have a circular, oval, or polygon cross sectional shape. The dimension of the core 18 as shown in its nonexpanded state of FIG. 1 is generally equal in diameter to, and slightly longer than the frame 16 in its collapsed state. It will be understood that this is but one embodiment of the initial, nonexpanded dimensions of the core 18. Other embodiments of the initial, nonexpanded dimensions of the core 18 include lengths shorter than the frame 16 in either its collapsed or extended state or lengths generally equal to either the frame 16.

The length, diameter and shape of the core 18 are selected according to the vessel and duration of occlusion so that the short and long term occlusion requirements for the desired treatment are met. Accordingly, different lengths, diameters and shapes can be selected for a particular treatment.

In an embodiment as shown in FIG. 4, a swelling control coating 23 covers the core interior portion 21 and controls the rate of expansion of the core interior portion 21. In an embodiment, the coating 23 reduces the rate of core expansion by about 50%. In an embodiment, the coating 25 reduces the rate of core expansion by about 25% or by about 33%. Swelling control coating 23 may cover the entire core interior portion 21. In another embodiment, coating 23 covers part of the core interior portion.

In an embodiment, the swelling control portion 21 is semipermeable to the substance that causes the interior core portion 21 to expand. Examples of the swelling control coating 23 include gelatins. Other examples of the swelling control coating 23 include materials that are biocompatible and are semipermeable to allow moisture to contact the core interior portion. In the case of hydrophillic core interior portions, the swelling control coating 23 impedes but does not completely bar moisture from coming into contact with and being absorbed by the hydrophillic core interior 21. In an embodiment, the coating 23 is significantly thinner than the interior portion 21, e.g., less than a tenth of the thickness of the interior portion.

The occlusion system 10 includes the delivery unit 14, which includes a catheter 31 that is inserted into the vessel. The catheter 31 typically is inserted into a vessel of a living being distally of the site at which the occlusion device 12 will be placed. For example, the catheter 31 can be positioned using the Seldinger technique or other similar surgical techniques. However, the placement of the catheter 31 may follow a circuitous path to arrive near the occlusion site. For example, if the occlusion site is in the head, the catheter 31 is inserted into a vessel system below the neck and threaded through the vessel system to the occlusion site.

Once the distal end 33 of the catheter 31 is adjacent to the occlusion site, the occlusion device 12 is inserted into the proximal end of the catheter 31. A proximal delivery wire 34 and a distal delivery wire 35 are respectively connected to the proximal and distal ends of the frame 16. The distal ends of wires 34 and 35 are spaced from each other to hold the frame 16 in its collapsed state (FIG. 1). Once the occlusion device 12 is positioned at the occlusion site, one of the proximal or distal delivery wires 34 or 35 is released from the frame 16 (FIG. 2). Only releasing one of the delivery wires allows the surgeon to hold the frame in place at the occlusion site while the frame expands and engages the vessel wall to securely anchor the occlusion device 12 at the occlusion site.

In the embodiment shown in FIG. 2, the distal delivery wire 35 has been released and the frame 16 is shown in its non-collapsed state that anchors the frame and expandable core 18 to the vessel wall. The other delivery wire, shown as delivery wire 34 in the FIG. 2 embodiment, is released from the frame 16 after the frame is anchored to the vessel (FIG. 3).

With the occlusion device 12 anchored at the occlusion site by frame 16, the core 18 expands or swells. In an embodiment, the coating 23 restricts the rate of expansion by the core interior portion 21. This serves two purposes. First, the core interior portion 21 expands slowly at the beginning of the surgical procedure to insert the occlusion device 12. Accordingly, the surgeon has ample time to position the occlusion device at the occlusion site. One example of the time period for the core 18 to fully expand is about thirty minutes from exposure to a catalyst such as water, air, bodily fluids and the like. Another example of core expansion time is in the order about 10 minutes. Another example of core expansion time is about an hour. The core interior portion 21 may expand due to contact with fluids in the vessel. In another embodiment, the core interior portion 21 is self-expanding regardless of its environment and is limited in its growth rate by the coating 23.

Second, the coating 23 does not expand with the core interior portion 21, thus the rate of expansion may increase as the volume of the core interior portion 21 increases. This is due to the coating 23 covering less surface area of the core interior portion 21 as it grows. Accordingly, in this embodiment, the rate of expansion of the interior portion 18 increases after it is positioned at the occlusion site.

FIG. 3 shows the occlusion device 12 positioned at an occlusion site of a vessel with the frame 16 engaged on an interior wall of a vessel 20 and the core 18 swelled to occlude the vessel. The occlusion device 12 with the core 18 expanded to its occlusion size in an embodiment of the present invention has a length of less than one centimeter. In one embodiment, the occlusion device 12 has a length of about five millimeters. In an embodiment, the length of the occlusion device 12 is less than five millimeters. In an embodiment, the length of the occlusion device 12 is less than about three millimeters. In an embodiment, the length of the occlusion device 12 is less than three millimeters. Consequently, the occlusion device 12 precisely occludes a vessel due to its relatively short length compared to some known balloon type occlusion systems. Some conventional balloon type occlusion systems have a length of one to three centimeters. FIG. 4 shows one advantage of the present occlusion device 12. Occlusion device 12 has a shorter length than balloon type occlusion systems. This allows the present occlusion device 12 to block fluid flow in the desired vessel but allow fluid flow in other feeder vessels, such as branch vessels 42 and 43 fluidly connected to the vessel 20 upstream of the occlusion device 12. The short length of the occlusion device 12 reduces the number of feeder vessels that are blocked by the occlusion device 12.

In some treatments, it is desirable to occlude additional feeder vessels, for example when both the main vessel 20 and at least one additional feeder vessel, e.g., vessel 42 in FIG. 6, feed the treatment area. The treatment area in FIG. 6 is a tumor 60 schematically shown as receiving blood from vessels 20 and 42. In this case, a plurality of occlusion devices 12A and 12B are implemented end-to-end in the main vessel 20. The frames of the occlusion devices 12A and 12B may be chained together or in close proximity to each other. Both occlusion devices 12A and 12B occlude main vessel 20. The proximal occlusion device 12A also occludes the feeder vessel 42. FIG. 6 shows an embodiment with two occlusion devices. Other embodiments may have greater than two occlusion devices.

In an embodiment, the distal delivery wire 35 is tubular with a hollow interior to deliver a chemotherapeutic agent into the vessel just past the occlusion site before wire 35 is released from the occlusion device 12. This applies the chemotherapeutic just prior to occlusion to enhance precision of drug treatment and drug retention. Thus, the present invention provides precisely targeted chemotherapeutic treatment as well as ischemic treatment distally from the occlusion device. Such a treatment may be well suited for treatment of abnormal tissue growth such as tumors.

In an embodiment, the occlusion device 12 includes a chemotherapeutic agent or drug that releases from the occlusion device to assist in treatment. The chemotherapeutic agent may be included on the frame 16, for example, by bonding a releasable chemotherapeutic agent to a surface of at least one wire of frame 16. The chemotherapeutic agent may be included in the core 18, in either or both of the interior portion 21 or coating 23. That is, the interior portion, coating, or both are loaded with the chemotherapeutic agent, which is released from the core 18 to provide treatment at the occlusion site or downstream of the occlusion site. Thus, the chemotherapeutic agent is supplied by the occlusion device 12 either by itself or in conjunction with another source of a chemotherapeutic agent.

In an embodiment, the chemotherapeutic agent is applied at or distally of the occlusion site. In an embodiment, the chemotherapeutic agent is applied using conventional methods prior to insertion of the occlusion device. In an embodiment, the chemotherapeutic agent is supplied through at least one of the delivery wires 34 or 35. Such a delivery wire is tubular such that the chemotherapeutic agent travels through the interior of the at least one delivery wire 34 or 35 and exits an open distal end of the wire. Accordingly, the chemotherapeutic agent is injected into the patient generally close to the desired treatment site, which may improve the efficacy of the chemotherapeutic agent, reduce the required dosage, reduce side effects, or provide other benefits to the living being being treated.

In an embodiment of the invention, it may be desired to provide a chemotherapeutic agent over a period of time or after the vessel is occluded. A chemotherapeutic agent delivery catheter 70 (FIG. 7) is inserted into the vessel 20. Catheter 70 is threaded through the vessel until it comes into contact with the core 18. Force is applied to the catheter 70. Core 18 yields to the catheter. Thus, the catheter 70 punctures through the core 18 such that a distal, delivery end 71 of the catheter 70 extends past a distal end of the occlusion device 12. The core 18 is made of a material that occludes the vessel by preventing fluid flow through the vessel but is pierced by catheter 70. The chemotherapeutic agent is then transferred through the catheter 70 and precisely applied to a treatment site distal the occlusion device 12. If the treatment regimen requires frequent or accurate dosing requirements, the catheter 70 may remain through the core 18 for a period of time, such as hours or days. In other treatment regimens, including more infrequent dosing or a low number of doses, the catheter 70 is inserted into the vessel 20 and punctures through the core 18 to deliver the chemotherapeutic agent distally or downstream of the occlusion device 12. Thereafter, the catheter 70 is removed from the core 18 and vessel 20. In an embodiment, the expandable nature of the core 18 results in it closing the aperture therein created by the catheter 70. Thus, the device 12 re-occludes the vessel 20.

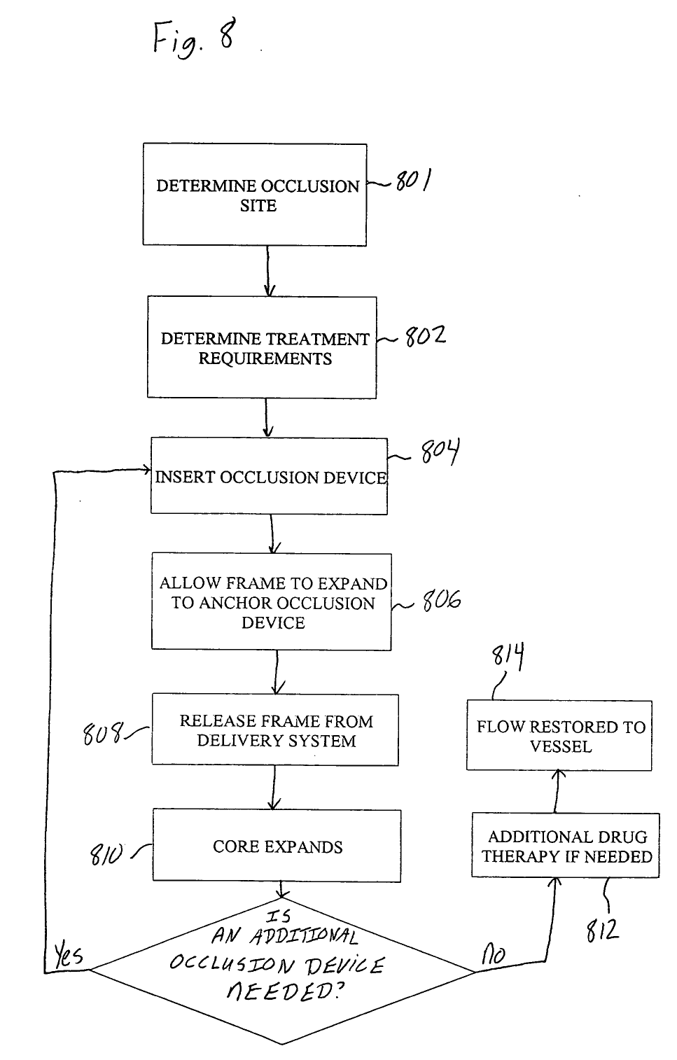

Referring now to FIG. 8, one method of the present invention begins with determining the occlusion site (801). Factors for determining the occlusion site include size of the vessel that can be occluded by an occlusion device 12, the vessels providing a fluid to the treatment area, and any adjacent feeder vessel which does not feed the treatment area. It is desirable to minimize the occlusion of adjacent branch vessels that do not provide fluid to the treatment site. A further step 802 is determining the treatment requirements, which includes ischemic treatment only, drug therapy followed by ischemic treatment, ischemic treatment followed by drug therapy, simultaneous ischemic treatment and drug therapy, and any of the preceding with additional drug applications, for example, chemotherapeutic agent(s) released from the occlusion device 12, catheters 70 providing additional chemotherapeutic agent(s), or a combination of both. Such a determination assists in selecting an appropriate occlusion device 12. The selected occlusion device 12 is inserted into vessel (804). One procedure for inserting the occlusion device 12 is to make an incision in the living being remote the occlusion site and using delivery unit 14, the occlusion device is threaded through a vessel system, for example, the circulatory system, until it is positioned at the occlusion site. Any number of methods may be employed to position the occlusion device. One method is to provide the frame with a radiopaque material, for example on the frame 16, and track the position of the occlusion device using X-Ray equipment, fluoroscopy, endoscopic viewing equipment, or other non-invasive viewing apparatus.

Once the occlusion device is correctly positioned, the frame 16 is allowed to expand (806). In an embodiment, the frame is held in place during its expansion so that it does not shift longitudinally in the vessel while the frame is expanding. The frame is then released from the delivery system (808). The core 18, which is the element of the occlusion device 12 that occludes the vessel, begins to expand if it has not already begun to do so. In an embodiment, the core 18 begins to expand as soon as it is inserted into the vessel. As discussed above, the core 18 may include coating 23 that restricts, but does not prevent, the expansion of the core 18. In an embodiment, the removal of the delivery unit 14 from the occlusion device 12 triggers core expansion. If another occlusion device 12 is needed, then the method returns to step 804 and proceeds as discussed herein. If no other occlusion device is needed, then additional drug therapy is provided on an as needed basis (812). For example, the occlusion device 12 may release additional chemotherapeutic agent(s) and/or additional chemotherapeutic agent(s) may be administered to the living being, either endemically or generally administered. Endemic administration includes the use of catheter 70 as described herein.

The occlusion device 12, after a period of time, stops occluding the vessel. Fluid flow is then restored to the vessel (814). In an embodiment, at least part of the occlusion device 12 degrades inside the vessel to restore flow. Thereafter, the remaining part of the occlusion device may be retrieved using a method such as surgical removal. The remaining part may continue to reside in the vessel to support it while flow is restored in the vessel. In another embodiment, the occlusion device is surgically removed.

The occlusion system of the present invention is suited for use in treating a variety of medical conditions. Examples of such conditions include tumors, high vascular flow malformations such as fistulas and AVM, and other forms of brain attacks.

It is apparent from the present disclosure that the expansion rate of the vessel occluding core 18 does not adversely effect the positioning of the occlusion device 12 as the frame 16 anchors the occlusion device to the vessel prior to occlusion taking place. The frame 16 expands first and faster than the core 18. The core 18 and not the frame 16 occludes the vessel.

The aforementioned description is not to be interpreted to exclude other occlusion devices advantageously employing the present invention. Other embodiments may be desired by those skilled in the art without departing from the spirit and scope of the present invention.

What is claimed is:

1. A device for occluding a vessel in a living being, comprising:
a frame comprising a collapsed transport state and a non-collapsed anchor state; and
an expansion element connected to the frame, the expansion element comprising a first state and an second state larger than the first state, the expansion element in the first state being transportable through a vessel or duct, the expansion element in the second, enlarged state blocking the vessel.

2. The device of claim 1, wherein the frame is comprised of a first self expandable elements.

3. The device of claim 2, wherein the expansion element includes a second self expandable material.

4. The device of claim 3, wherein the second self expandable material comprises a core that includes at least one of a hydrogel and a polyurethane.

5. The device of claim 4, wherein the second self expandable material further comprises a coating on the core, wherein the coating limits the rate of expansion by the core.

6. The device of claim 5, wherein the coating includes a therapeutic.

7. The device of claim 5, wherein the coating is a gelatin.

8. The device of claim 4, wherein the core is biodegradable and after an occlusion time period the core dissolves and the vessel is no longer blocked.

9. The device of claim 2, wherein the frame includes an expandable stent, the stent having a collapsed state for travel through a vessel and an expanded state fixed on the vessel or duct, and the stent does not completely block the vessel.

10. The device of claim 9, wherein the stent includes a wire body, which is self-expandable.

11. The device of claim 1, wherein the frame does not occlude the vessel and the expansion element occludes the vessel.

12. The device of claim 1, wherein the expansion element is a colloid.

13. The device of claim 1, wherein the expansion element is a solid.

14. The device of claim 1, wherein the frame has a length of less than one centimeter in its non-collapsed state.

15. The device of claim 1, wherein the frame has a length of less than about five millimeters in its non-collapsed state.

16. The device of claim 1, wherein the core has a length of less than one centimeter in its expanded state.

17. The device of claim 1, wherein the core has a length of less than about five millimeters in its non-collapsed state.

18. A vessel occlusion system, comprising:
a transport unit; and
an occlusion device releasably connected to the delivery unit, the occlusion device including:
a frame including a collapsed transport state and a non-collapsed anchor state; and
an expansion element connected to the frame, the expansion element including a small state and an enlarged state, the expansion element in the small state being transportable through a vessel or duct, the expansion element in the enlarged state occluding the vessel.

19. The vessel occlusion system of claim 18, further comprising a drug delivery unit.

20. The vessel occlusion system of claim 19, wherein the drug delivery unit includes a catheter that extends through the expansion element in its enlarged state.

21. The vessel occlusion system of claim 19, wherein the drug delivery unit includes a chemotherapeutic agent releasable from at least one of the frame and the expansion element.

22. The vessel occlusion system of claim 18, wherein the occlusion device has a length of less than about five millimeters with the frame in its non-collapsed state and the expansion element in the enlarged state.

23. The vessel occlusion system of claim 18, wherein the expansion element includes at least one of a hydrogel and a polyurethane.

24. The vessel occlusion system of claim 18, wherein the expansion element is biodegradable and after an occlusion time period the expansion element degrades and the vessel is no longer occluded.

25. A vessel occlusion system for occluding multiple pathways, comprising a first occlusion device and a second occlusion device, wherein at least one of the first and second occlusion device includes:
a frame including a collapsed transport state and a non-collapsed anchor state; and
an expansion element connected to the frame, the expansion element including a small state and an enlarged state, the expansion element in the small state being transportable through a vessel or duct, the expansion element in the enlarged state blocking the vessel.

26. A method for occluding a vessel, comprising:
positioning an occlusion device in the vessel;
expanding a frame of the occlusion device to anchor the occlusion device to the vessel without occluding the vessel;
expanding a core of the occlusion device to occlude the vessel.

27. The method of claim 26, wherein expanding the frame occurs faster than expanding the core.

28. The method of claim 26, wherein expanding the core includes absorbing a fluid into a hydrophillic portion of the core.

29. The method of claim 26, wherein the core includes one of a hydrogel and a polyurethane.

30. The method of claim 26, further comprising allowing the core to biodegrade to restore flow in the vessel after a period of time.
